(19) United States
(12) Patent Application Publication
(10) Pub. No.: US 2022/0370375 A1
(43) Pub. Date: Nov. 24, 2022

(54) PAIN RELIEVING PATCH COMPRISING A PROPOLIS COMPOSITION AND ASSOCIATED METHODS

(71) Applicant: Leonid Radomyshelsky, San Diego, CA (US)

(72) Inventor: Leonid Radomyshelsky, San Diego, CA (US)

(21) Appl. No.: 17/749,424

(22) Filed: May 20, 2022

Related U.S. Application Data
(60) Provisional application No. 63/205,657, filed on May 21, 2021.

(51) Int. Cl. A61K 9/70

(52) U.S. Cl. CPC A61K 9/7076

(57) ABSTRACT

A patch for at least partially reducing at least one of pain, swelling, inflammation and irritation in at least one body part of a user is provided. The patch includes at least one cloth layer having the propolis composition embedded therein. The patch includes a first plastic layer disposed immediately against a first side of the at least one cloth layer, and a second plastic layer disposed immediately against a second side of the at least one cloth layer opposite the first side, such that the at least one cloth layer is sandwiched between the first and second plastic layers. The propolis composition is configured to at least partially reduce at least one of perceived pain, swelling, inflammation and irritation when the patch is applied to the at least one body part. Related methods of use and/or manufacture for such a patch are also provided.

### RELATED APPLICATIONS

This application claims priority to U.S. Provisional Application No. 63/205,657 filed on May 21, 2021, the entire contents of which are hereby incorporated by reference.

### BACKGROUND

There are a variety of compositions and associated methods commonly used to relieve body aches and pains, for example, hip pain, back pain, sinus pain, tooth pain, etc. The use of both over-the-counter and prescription nonsteroidal medications has been recommended for the treatment of pain. However, persistent long-term use of such medications presents safety concerns, especially when prescribing those medications for chronic and degenerative pain conditions. Patients and care providers are increasingly looking toward new compositions, techniques and associated methods of relieving such body aches and pains. Accordingly, there is a need for new compositions and methods of relieving body aches and pains.

The human body's natural response to injury results in inflammation-induced pain, swelling, and erythema. In order to reduce pain, anti-inflammatory agents such as NSAIDs act on the multiple inflammatory pathways, which, although often very effective, can have undesirable side effects including gastric ulceration, myocardial infarction, and stroke.

For centuries, natural anti-inflammatory compounds have been used to mediate the inflammatory process and often with fewer side effects. There remains a need for natural alternatives to address both chronic and sub-acute pain

It should be noted that this Background is not intended to be an aid in determining the scope of the claimed subject matter nor be viewed as limiting the claimed subject matter to implementations that solve any or all of the disadvantages or problems presented above. The discussion of any technology, documents, or references in this Background section should not be interpreted as an admission that the material described is prior art to any of the subject matter claimed herein.

### SUMMARY

In one implementation, a patch having a propolis composition embedded therein for at least partially reducing at least one of perceived pain, swelling, inflammation and irritation in at least one body part of a user is provided. The patch includes at least one cloth layer having the propolis composition embedded therein. The patch includes a first plastic layer disposed immediately against a first side of the at least one cloth layer. The patch includes a second plastic layer disposed immediately against a second side of the at least one cloth layer opposite the first side, such that the at least one cloth layer is sandwiched between the first and second plastic layers. The propolis composition is configured to at least partially reduce at least one of pain, swelling, inflammation and irritation in at least one body part of the user when the patch is applied to the at least one body part.

In another implementation, a method of using a patch having a propolis composition embedded therein for at least partially reducing at least one of perceived pain, swelling, inflammation and irritation in at least one body part of a user is provided. The method includes applying the patch to the at least one body part. The patch includes at least one cloth layer having the propolis composition embedded therein. The patch includes a first plastic layer disposed immediately against a first side of the at least one cloth layer. The patch includes a second plastic layer disposed immediately against a second side of the at least one cloth layer opposite the first side, such that the at least one cloth layer is sandwiched between the first and second plastic layers and the first plastic layer is disposed directly against the at least one body part of the user. The propolis composition is configured to at least partially reduce at least one of pain, swelling, inflammation and irritation in the at least one body part of the user when the first plastic layer is disposed directly against the at least one body part of the user.

In another implementation, a method of manufacturing a patch having a propolis composition embedded therein for at least partially reducing at least one of perceived pain, swelling, inflammation and irritation in at least one body part of a user is provided. The method includes soaking at least one cloth layer in a propolis composition for a predetermined period of time to form at least one cloth layer having the propolis composition embedded therein. The method includes disposing a first plastic layer immediately against a first side of the at least one cloth layer. The method includes disposing a second plastic layer immediately against a second side of the at least one cloth layer opposite the first side, such that the at least one cloth layer is sandwiched between the first and second plastic layers. The propolis composition is configured to at least partially reduce at least one of pain, swelling, inflammation and irritation in the at least one body part of the user when the patch is disposed directly against the at least one body part of the user.

It is understood that various configurations of the subject technology will become apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

Various embodiments are discussed in detail in conjunction with the Figures described below, with an emphasis on highlighting the advantageous features. These embodiments are for illustrative purposes only and any scale that may be illustrated therein does not limit the scope of the technology disclosed. These drawings include the following figures, in which like numerals indicate like parts.

FIG. 1A shows a top view of a patch having a propolis composition embedded therein, in accordance with some embodiments;

FIG. 1B shows a top view of another patch having a propolis composition embedded therein, in accordance with some embodiments;

FIG. 1C shows a cutaway view of either of the patches of FIGS. 1A and 1B, for example as viewed in direction of the arrows along the cut-line A-A′ of FIG. 1A or along the cut-line B-B′ of FIG. 1B, in accordance with some embodiments;

FIG. 2 shows a patch having a propolis composition embedded therein disposed on at least one of a nose and a forehead of a user, in accordance with some embodiments;

FIG. 3 shows a patch having a propolis composition embedded therein disposed on a jaw of a user, in accordance with some embodiments;

FIG. 4 shows a patch having a propolis composition embedded therein disposed on a low back of a user, in accordance with some embodiments;

FIG. 5 shows a patch having a propolis composition embedded therein disposed on a hip of a user, in accordance with some embodiments;

FIG. 6 shows a flowchart relating to a method of using a patch having a propolis composition embedded therein, in accordance with some embodiments; and

FIG. 7 shows another flowchart relating to a method of manufacturing and/or preparing a patch having a propolis composition embedded therein, in accordance with some embodiments.

### DETAILED DESCRIPTION

The following description and examples illustrate some exemplary implementations, embodiments, and arrangements of the disclosed invention in detail. Those of skill in the art will recognize that there are numerous variations and modifications of this invention that are encompassed by its scope. Accordingly, the description of a certain example embodiments should not be deemed to limit the scope of the present invention.

Implementations of the technology described herein are directed generally to a patch having a propolis composition embedded therein. Pain, heat, redness, and swelling are associated with the inflammatory process. Abnormalities of the joints, associated muscles, tendons, ligaments, organs, skin, and bones can result in pain. The patch and/or methods described herein simultaneously can address a variety of medical conditions, at least in part, by using embedded all-natural propolis to at least partially reduce pain, swelling, inflammation and/or irritation in at least one body part or portion of the body of a user.

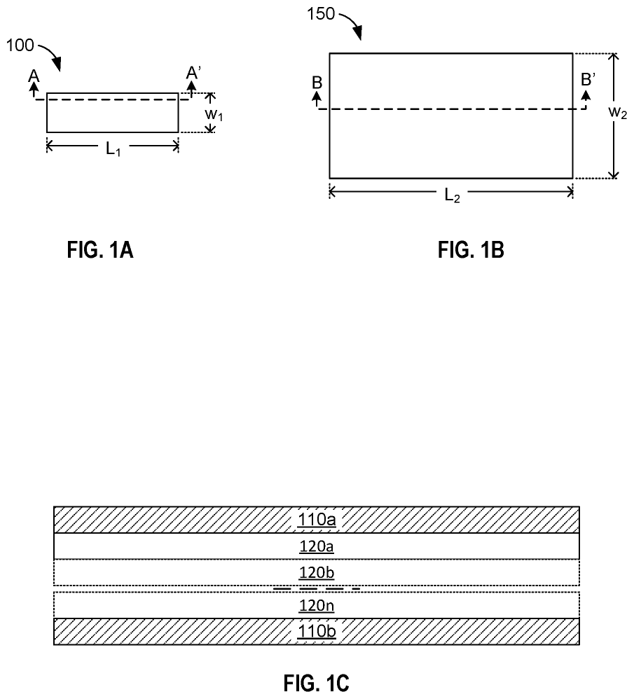

Propolis, also known as bee glue, is a wax-like substance collected by honeybees, from flowers, to protect and repair their hives. Propolis generally comprises phenol acids, flavonoids, terpenes, fatty acids, stilbenes and β-steroids, among other component substances. Extracts of propolis can possess antimicrobial activity, anti-inflammatory activity and, in some cases, antitumor effects in some cancer cells. The present disclosure describes several embodiments of a patch in which a propolis composition is embedded therein for use in at least partially reduce perceived pain, swelling, inflammation and/or irritation in at least one body part or portion of the body of a user, in connection with FIGS. 1A-7. As disclosed herein, a homeopathic, natural alternative to conventional pain relievers such as non-steroidal anti-inflammatory medications and antibiotics is provided.

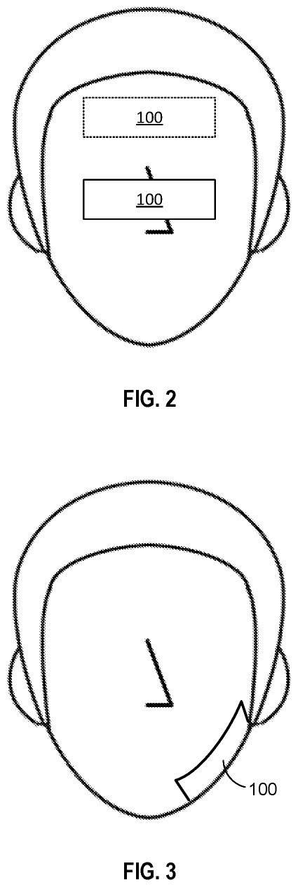

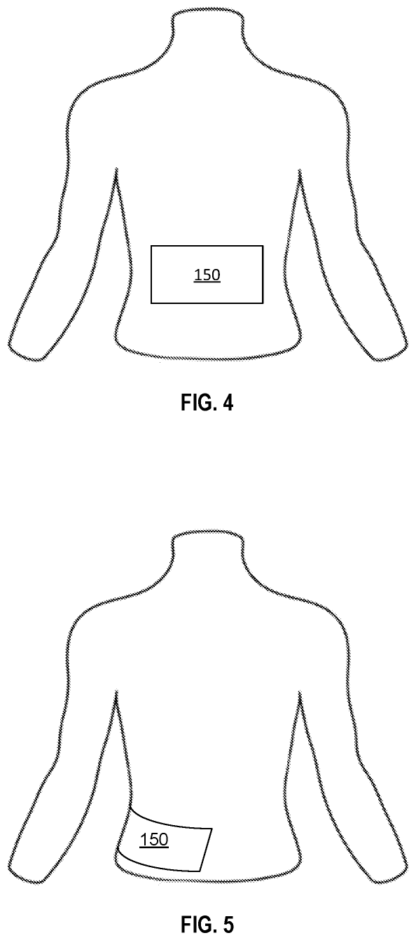

FIG. 1A shows a top view of a patch 100 having a propolis composition embedded therein, in accordance with some embodiments. Patch 100 can be a small patch having a first length L₁ and a first width w₁, each sized such that patch 100 is configured to be applied and/or otherwise disposed on relatively smaller areas of the body, for example across the bridge of the nose or on a portion of the forehead (see, e.g., FIG. 2), or on a portion of a jaw (see, e.g., FIG. 3). For example, first length L₁ can be 2.5 inches, while first width w₁ can be 1 inch. While patch 100 is illustrated as having a substantially rectangular shape, the present disclosure is not so limited and patch 100 can have any suitable shape, for example, having rounded corners, tapered portions along one or both of a length and/or a width of patch 100, and/or having a substantially circular, ovoid, triangular or any other suitable shape. Applying patch 100 to such relatively smaller areas of the body can provide an all-natural, external and/or topical treatment for the alleviation of pain, infection, inflammation and/or swelling associated with one or more maladies, e.g., headaches, allergies, sinus infections, tooth and/or jaw aches at, adjacent to, or near those relatively smaller areas of the body. Specifics of patch 100 will be described in more detail in connection with at least FIG. 1C below.

FIG. 1B shows a top view of another patch 150 having a propolis composition embedded therein, in accordance with some embodiments. Patch 150 can be a large patch having a second length L₂ and a second width w₂, each sized such that patch 150 is configured to be applied and/or otherwise disposed on relatively larger areas of the body, for example on a low back (see, e.g., FIG. 4) or hip of the user (see, e.g., FIG. 5). For example, second length L₂ can be 4.5 inches, while second width w₂ can be 4 inches. In some embodiments, second length L₂ is greater than first length L₁ and second width w₂ is greater than first width w₁. While patch 150 is illustrated as having a substantially rectangular shape, the present disclosure is not so limited and patch 150 can have any suitable shape, for example, having rounded corners, tapered portions along one or both of a length and/or a width of patch 150, and/or having a substantially circular, ovoid, triangular or any other suitable shape. Applying patch 150 to such relatively larger areas of the body can provide an all-natural, external and/or topical treatment for the alleviation of pain, infection, inflammation and/or swelling associated with one or more maladies, e.g., back pain, hip pain at, adjacent to, or near those relatively larger areas of the body. Specifics of patch 150 will be described in more detail in connection with at least FIG. 1C below.

FIG. 1C shows a cutaway view of either of the patches 100, 150 of FIGS. 1A and 1B, for example as viewed in direction of the arrows along the cut-line A-A′ of FIG. 1A or along the cut-line B-B′ of FIG. 1B, in accordance with some embodiments. Patch 100, 150 includes at least one layer of cloth 120a, 120b . . . 120n having a propolis composition embedded therein. Cloth layer(s) 120a-120n can comprise any suitable cloth or woven material, for example, synthetic cloth, cotton cloth, or in a preferred example, linen. In some embodiments, the one or more cloth layer(s) 120a-120n can be embedded with the propolis composition by soaking the cloth layers 120a-120n in the propolis composition for a predetermined length of time. In some embodiments, the propolis composition can have a potency of approximately 93% propolis and approximately 7% alcohol. In some embodiments, the propolis composition is widely available and can be commercially obtained from, for example, Ebay®, Amazon^{T}®, etc. In some embodiments, cloth layers 120a-120n can be soaked in the propolis composition for a predetermined time period of approximately 30 seconds.

In some embodiments, patch 100, 150 includes a single layer of cloth 120a having a propolis composition embedded therein. In some other embodiments, patch 100, 150 includes two stacked and/or folded layers of cloth 120a, 120b, each having a propolis composition embedded therein. In yet other embodiments, patch 100, 150 includes three or more stacked and/or folded layers of cloth 120a-120n, each having a propolis composition embedded therein. In some embodiments, a potency of the propolis composition increases as the number of layers of cloth 120a-120n in patch 100, 150 increases. In some but not all embodiments, the increase in potency of the propolis composition as the number of layers of cloth 120a-120n increases is a byproduct of stacking multiple layers of cloth 120a-120n, each having substantially the same potency of propolis composition embedded therein, rather than by increasing the potency of the propolis composition in any single cloth layer 120a-120n. However, the present disclosure is not so limited and one or more of the layers of cloth 120a-120n can be configured with or to have a different potency of propolis composition embedded therein compared to at least one other of the layers of cloth 120a-120n by any known or unknown method, e.g., soaking one or more of the cloth layers 120a-120n in a more potent propolis composition and/or by soaking the one or more of the cloth layers 120a-120n in the propolis composition having a particular potency for a longer period of time.

Patch 100, 150 further includes a first plastic layer 110a disposed immediately against a first side of the one or more layers of cloth 120a-120n and a second plastic layer 110b disposed immediately against a second side of the one or more layers of cloth 120a-120n that is opposite the first side. In this way, the one or more layers of cloth 120a-120n are sandwiched between first and second plastic layers 110a, 110b of patch 100, 150. In some embodiments, patch 100, 150 does not have a predetermined front or back surface, e.g., patch 100, 150 can be applied with either of first or second plastic layer 110a, 110b disposed directly against the skin of the user. In some embodiments, patch 100, 150 is configured to be worn during the day or overnight, while the user is awake or sleeping, or any combination thereof.

In some embodiments, one or more components of the propolis composition, e.g., constituent oils, etc., can at least partially migrate through micropores in the one or more layers of cloth 120a-120n and, in some embodiments, also through micropores in one or both of first and second plastic layers 110a, 110b. In some such embodiments where one or more components of the propolis composition, e.g., constituent oils, etc., can at least partially migrate through micropores in one or both of first and second plastic layers 110a, 110b, at least those one or more components of the propolis composition can also provide a secondary function, unrelated to the direct treatment of pain, infection, inflammation and/or swelling associated with one or more maladies, as an adhesive for removably attaching patch 100, 150 to the wearer, user and/or patient without the use of additional adhesive.

Several example applications and/or uses of patch 100, 150 will now be described in connection with FIGS. 2-5.

FIG. 2 shows patch 100 having a propolis composition embedded therein disposed on at least one of a nose and a forehead of a user, in accordance with some embodiments. For example, a user can apply patch 100 across the bridge of his or her nose (see solid-lined patch 100) and/or on his or her forehead (see dotted-lined patch 100) for a period of time of between 30 seconds and 5 minutes and subsequently experience a reduction in or alleviation of pain, infection, inflammation and/or swelling associated with one or more maladies, e.g., headaches, allergies, sinus infections at adjacent to, or near the user's nose, forehead and/or sinus cavities.

FIG. 3 shows patch 100 having a propolis composition embedded therein disposed on a jaw of a user, in accordance with some embodiments. For example, a user can apply patch 100 to a portion of his or her jaw for a period of time of between 20 seconds and 5 minutes and subsequently experience a reduction in or alleviation of pain, infection, inflammation and/or swelling associated with one or more maladies, e.g., jaw or toothache adjacent to, or near the user's jaw.

FIG. 4 shows patch 150 having a propolis composition embedded therein disposed on a low back of a user, in accordance with some embodiments. For example, a user can apply patch 150 to his or her low back for a period of time of between 30 seconds and 5 minutes and subsequently experience a reduction in or alleviation of pain, infection, inflammation and/or swelling associated with one or more maladies, e.g., pain adjacent to or near the user's low back.

FIG. 5 shows patch 150 having a propolis composition embedded therein disposed on a hip of a user, in accordance with some embodiments. For example, a user can apply patch 150 to his or her hip area for a period of time of between 30 seconds and 5 minutes and subsequently experience a reduction in or alleviation of pain, infection, inflammation and/or swelling associated with one or more maladies, e.g., pain adjacent to or near the user's hip.

While FIGS. 2-5 illustrate several example uses and/or applications of patch 100, 150, the present disclosure is not so limited and patch 100, 150 can be used on any body part for similar reductions and/or alleviations of pain, infection, inflammation and/or swelling.

Discussion will now turn to example methods for using, manufacturing and/or otherwise preparing a patch having a propolis composition embedded therein, for example as described in connection with any of at least FIGS. 1A-5 above.

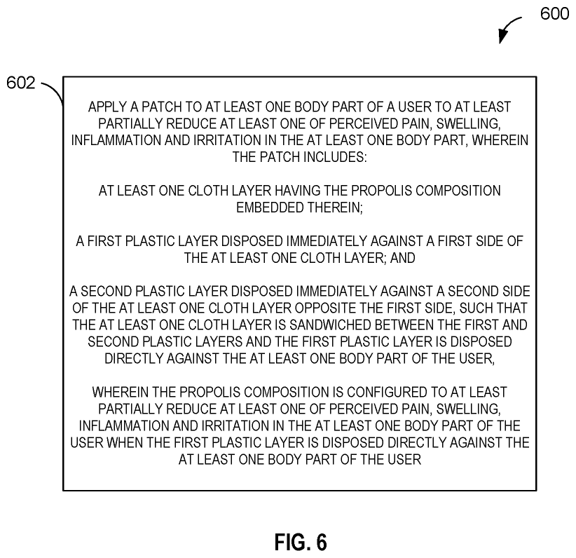

FIG. 6 shows a flowchart 600 relating to a method of using a patch having a propolis composition embedded therein for at least partially reducing at least one of perceived pain, swelling, inflammation and irritation in at least one body part of a user, in accordance with some embodiments. Such methods comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

Block 602 of flowchart 600 includes applying the patch to the at least one body part of a user to at least partially reduce at least one of perceived pain, swelling, inflammation and irritation in the at least one body part. For example, as previously described in connection with at least FIGS. 1A-5, patch 100, 150 can include at least one cloth layer 120a-120n having the propolis composition embedded therein. Patch 100, 150 can further include a first plastic layer 110a disposed immediately against a first side of the at least one cloth layer 120a-120n. Patch 100, 150 can further include a second plastic layer 110b disposed immediately against a second side of the at least one cloth layer 120a-120n opposite the first side, such that the at least one cloth layer 120a-120n is sandwiched between first and second plastic layers 110a, 110b. In some such embodiments, first plastic layer 110a is disposed directly against the skin of at least one body part of the user. However, it is appreciated that second plastic layer 110b can alternatively be disposed directly against the skin of at least one body part of the user. As previously described, the propolis composition is configured to at least partially reduce at least one of perceived pain, swelling, inflammation and irritation in the at least one body part of the user when the first plastic layer (or second plastic layer) is disposed directly against the at least one body part of the user.

In some embodiments, the at least one cloth layer comprises a single cloth layer 120a having the propolis composition embedded therein. In some embodiments, the at least one cloth layer comprises a plurality of stacked and/or folded cloth layers 120a-120b or 120a-120n, each having the propolis composition embedded therein. In some embodiments, the at least one cloth layer 120a-120n comprises one of a synthetic material, a cotton material and linen. In some embodiments, the propolis composition embedded in the at least one cloth layer 120a-120n has a propolis concentration of approximately 93% propolis and approximately 7% alcohol. In some embodiments, a propolis concentration of the at least one cloth layer increases with increasing number of cloth layers in the at least one cloth layer. In some embodiments, each of the plurality of stacked cloth layers 120a-120n has a same concentration of the propolis composition embedded therein. In some embodiments, at least one of the plurality of stacked cloth layers (e.g., 120a) has a greater concentration of the propolis composition embedded therein compared to at least one other of the plurality of stacked cloth layers (e.g., any of 120b-120n). In some embodiments, patch 100 has a length L₁ of approximately 2.5 inches and a width w₁ of approximately 1 inch such that patch 100 is suitably configured to be disposed on at least one of a bridge of a nose of the user, a forehead of the user and a jaw of the user. In some embodiments, patch 150 has a length L₂ of approximately 4.5 inches and a width w₂ of approximately 4 inches such that patch 150 is suitably configured to be disposed on at least one of a low back of the user and a hip of the user.

In some embodiments, one or more components of the propolis composition are configured to migrate through micropores in the at least one cloth layer 120a-120n. In some embodiments, one or more components of the propolis composition are configured to migrate through micropores in at least one of the first and second plastic layers 110a, 110b. In some embodiments, the one or more components of the propolis composition function as an adhesive for removably attaching patch 100, 150 to the user in addition to at least partially reducing at least one of perceived pain, swelling, inflammation and irritation in the at least one body part of the user when patch 100, 150 is applied to the at least one body part.

In some embodiments, patch 100, 150 is configured to at least partially reduce at least one of perceived pain, swelling, inflammation and irritation in: at least one of a nose, a sinus or a head of the user when patch 100 is applied to at least one of a bridge of the nose and a forehead of the user; at least one of a tooth or a jaw of the user when patch 100 is applied to the jaw of the user; a low back of the user when patch 150 is applied to the low back of the user; and a hip of the user when patch 150 is applied to the hip of the user.

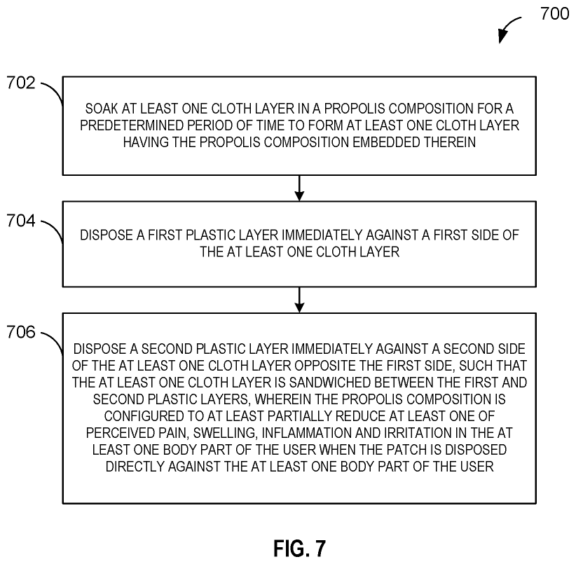

FIG. 7 shows another flowchart 700 relating to a method of manufacturing a patch having a propolis composition embedded therein for at least partially reducing at least one of perceived pain, swelling, inflammation and irritation in at least one body part of a user, in accordance with some embodiments. Such methods comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

Block 702 of flowchart 700 includes soaking at least one cloth layer in a propolis composition for a predetermined period of time to form at least one cloth layer having the propolis composition embedded therein. For example, as previously described in connection with at least FIGS. 1A-5, at least one cloth layer can be soaked in a propolis composition for a predetermined period of time to form at least one cloth layer 120a-120n having the propolis composition embedded therein.

Block 704 of flowchart 700 includes disposing a first plastic layer immediately against a first side of the at least one cloth layer. For example, as previously described in connection with at least FIGS. 1A-5, a first plastic layer 110a can be disposed immediately against a first side of the at least one cloth layer(s) 120a-120n.

Block 706 of flowchart 700 includes disposing a second plastic layer immediately against a second side of the at least one cloth layer opposite the first side, such that the at least one cloth layer is sandwiched between the first and second plastic layers, wherein the propolis composition is configured to at least partially reduce at least one of perceived pain, swelling, inflammation and irritation in the at least one body part of the user when the patch is disposed directly against the at least one body part of the user. For example, as previously described in connection with at least FIGS. 1A-5, a second plastic layer 110b can be disposed immediately against a second side of the at least one cloth layer(s) 120a-120n opposite the first side, such that the at least one cloth layer 120a-120n is sandwiched between the first and second plastic layers 110a, 110b. The propolis composition is configured to at least partially reduce at least one of perceived pain, swelling, inflammation and irritation in the at least one body part of the user when patch 100, 150 is disposed directly against the at least one body part of the user.

In some embodiments, the at least one cloth layer comprises a single cloth layer 120a having the propolis composition embedded therein. In some embodiments, the at least one cloth layer comprises a plurality of stacked and/or folded cloth layers 120a-120b or 120a-120n, each having the propolis composition embedded therein. In some embodiments, the at least one cloth layer 120a-120n comprises one of a synthetic material, a cotton material and linen. In some embodiments, the propolis composition embedded in the at least one cloth layer 120a-120n has a propolis concentration of 93%. In some embodiments, a propolis concentration of the at least one cloth layer increases with increasing number of cloth layers in the at least one cloth layer. In some embodiments, each of the plurality of stacked cloth layers 120a-120n has a same concentration of the propolis composition embedded therein. In some embodiments, at least one of the plurality of stacked cloth layers (e.g., 120a) has a greater concentration of the propolis composition embedded therein compared to at least one other of the plurality of stacked cloth layers (e.g., any of 120b-120n). In some embodiments, patch 100 has a length L₁ of approximately 2.5 inches and a width w₁ of approximately 1 inch such that patch 100 is suitably configured to be disposed on at least one of a bridge of a nose of the user, a forehead of the user and a jaw of the user. In some embodiments, patch 150 has a length L₂ of approximately 4.5 inches and a width w₂ of approximately 4 inches such that patch 150 is suitably configured to be disposed on at least one of a low back of the user and a hip of the user.

In some embodiments, one or more components of the propolis composition are configured to migrate through micropores in the at least one cloth layer 120a-120n. In some embodiments, one or more components of the propolis composition are configured to migrate through micropores in at least one of the first and second plastic layers 110a, 110b. In some embodiments, the one or more components of the propolis composition function as an adhesive for removably attaching patch 100, 150 to the user in addition to at least partially reducing at least one of perceived pain, swelling, inflammation and irritation in the at least one body part of the user when patch 100, 150 is applied to the at least one body part.

In some embodiments, patch 100, 150 is configured to at least partially reduce at least one of perceived pain, swelling, inflammation and irritation in: at least one of a nose, a sinus or a head of the user when patch 100 is applied to at least one of a bridge of the nose and a forehead of the user; at least one of a tooth or a jaw of the user when patch 100 is applied to the jaw of the user; a low back of the user when patch 150 is applied to the low back of the user; and a hip of the user when patch 150 is applied to the hip of the user.

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure. For example, a system or an apparatus may be implemented, or a method may be practiced using any one or more of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

Examples of Application/Use

A patch, as described anywhere herein, may be applied to any affected area of the body for reducing and/or eliminating pain, as needed. Upon application for between 40 seconds to one minute, for example, such patches can begin to provide pain relief relatively quickly, e.g., within 5 to 10 minutes, without any unexpected negative side-effects except, for example, where a person were allergic to bees and/or bee products. Several examples of patch application by real users follow.

A first user was suffering from back pain as a result of severed vertebrae caused by a motorcycle accident in 2012. Typically, this first user suffered back pain of approximately 6 out of 10, approximately 5 days a week, while working. The first user applied an appropriate patch, as described herein, to his back for 8 hours and began to experience pain relief within an hour of the onset of application. The first patient applied one of 3 patches, as needed, four separate times for 4-8 hours on each application, on different days and reported a reduction in the intensity of pain from a 6 out of 10 to approximately 2-3 out of 10. The first user has been applying patches, as needed, for the past 2 months and has reported no adverse side effects or allergies. Now, the first user applies a patch approximately 3 times per week, as needed when in pain.

A second user was suffering from neck pain. The second user applied an appropriate patch, as described herein, to his neck for approximately 30 minutes and experienced a significant reduction in neck pain within 20 minutes of application, from a single application. This second user has reported no adverse side effects or allergies.

### GENERAL INTERPRETIVE PRINCIPLES FOR THE PRESENT DISCLOSURE

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure. For example, a system or an apparatus may be implemented, or a method may be practiced using any one or more of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

With respect to the use of plural vs. singular terms herein, those having skill in the art can translate from the plural to the singular and/or from the singular to the plural as is appropriate to the context and/or application. The various singular/plural permutations may be expressly set forth herein for sake of clarity.

When describing an absolute value of a characteristic or property of a thing or act described herein, the terms “substantial,” “substantially,” “essentially,” “approximately,” and/or other terms or phrases of degree may be used without the specific recitation of a numerical range. When applied to a characteristic or property of a thing or act described herein, these terms refer to a range of the characteristic or property that is consistent with providing a desired function associated with that characteristic or property. For example, the terms “approximately,” “about” and “substantially” may refer to an amount that is within less than 10% of, within less than 5% of, within less than 1% of, within less than 0.1% of, and within less than 0.01% of the stated amount.

In those cases where a single numerical value is given for a characteristic or property, it is intended to be interpreted as at least covering deviations of that value within one significant digit of the numerical value given.

If a numerical value or range of numerical values is provided to define a characteristic or property of a thing or act described herein, whether or not the value or range is qualified with a term of degree, a specific method of measuring the characteristic or property may be defined herein as well. In the event no specific method of measuring the characteristic or property is defined herein, and there are different generally accepted methods of measurement for the characteristic or property, then the measurement method should be interpreted as the method of measurement that would most likely be adopted by one of ordinary skill in the art given the description and context of the characteristic or property. In the further event there is more than one method of measurement that is equally likely to be adopted by one of ordinary skill in the art to measure the characteristic or property, the value or range of values should be interpreted as being met regardless of which method of measurement is chosen.

It will be understood by those within the art that terms used herein, and especially in the appended claims (e.g., bodies of the appended claims) are intended as “open” terms unless specifically indicated otherwise (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

In those instances where a convention analogous to “at least one of A, B, and C” is used, such a construction would include systems that have A alone, B alone, C alone, A and B together without C, A and C together without B, B and C together without A, as well as A, B, and C together. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include A without B, B without A, as well as A and B together.”

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

What is claimed is:

1. A patch having a propolis composition embedded therein for at least partially reducing at least one of perceived pain, swelling, inflammation and irritation in at least one body part of a user, the patch comprising:
at least one cloth layer having the propolis composition embedded therein;
a first plastic layer disposed immediately against a first side of the at least one cloth layer; and
a second plastic layer disposed immediately against a second side of the at least one cloth layer opposite the first side, such that the at least one cloth layer is sandwiched between the first and second plastic layers,
wherein the propolis composition is configured to at least partially reduce at least one of pain, swelling, inflammation and irritation in at least one body part of the user when the patch is applied to the at least one body part.

2. The patch of claim 1, wherein the at least one cloth layer comprises a single cloth layer having the propolis composition embedded therein.

3. The patch of claim 1, wherein the at least one cloth layer comprises a plurality of stacked cloth layers, each having the propolis composition embedded therein.

4. The patch of claim 1, wherein the at least one cloth layer is constructed from one of a synthetic material, a natural material, or combinations thereof.

5. The patch of claim 4, wherein said natural material is linen.

6. The patch of claim 1, wherein the propolis composition embedded in the at least one cloth layer has a propolis concentration of approximately 93%.

7. The patch of claim 1, wherein a propolis concentration of the at least one cloth layer increases with increasing number of cloth layers in the at least one cloth layer.

8. The patch of claim 3, wherein each of the plurality of stacked cloth layers has a same concentration of the propolis composition embedded therein.

9. The patch of claim 3, wherein at least one of the plurality of stacked cloth layers has a greater concentration of the propolis composition embedded therein compared to at least one other of the plurality of stacked cloth layers.

10. The patch of claim 1, wherein the patch has a length of approximately 2.5 inches and a width of approximately 1 inch such that the patch is suitably configured to be disposed on at least one of a bridge of a nose of the user, a forehead of the user and a jaw of the user.

11. The patch of claim 1, wherein the patch has a length of approximately 4.5 inches and a width of approximately 4 inches such that the patch is suitably configured to be disposed on at least one of a low back of the user and a hip of the user.

12. The patch of claim 1, wherein one or more components of the propolis composition are configured to migrate through micropores in the at least one cloth layer.

13. The patch of claim 1, wherein one or more components of the propolis composition are configured to migrate through micropores in at least one of the first and second plastic layers.

14. The patch of claim 13, wherein the one or more components of the propolis composition function as an adhesive for removably attaching the patch to the user in addition to at least partially reducing at least one of perceived pain, swelling, inflammation and irritation in the at least one body part of the user when the patch is applied to the at least one body part.

15. The patch of claim 1, wherein the patch is configured to at least partially reduce at least one of pain, swelling, inflammation and irritation in:
at least one of a nose, a sinus or a head of the user when the patch is applied to at least one of a bridge of the nose and a forehead of the user;
at least one of a tooth or a jaw of the user when the patch is applied to the jaw of the user;
a low back of the user when the patch is applied to the low back of the user; and
a hip of the user when the patch is applied to the hip of the user.

16. A method of using a patch having a propolis composition embedded therein for at least partially reducing at least one of pain, swelling, inflammation and irritation in at least one body part of a user, the method comprising:
applying the patch to the at least one body part, wherein the patch comprises:
at least one cloth layer having the propolis composition embedded therein;
a first plastic layer disposed immediately against a first side of the at least one cloth layer; and
a second plastic layer disposed immediately against a second side of the at least one cloth layer opposite the first side, such that the at least one cloth layer is sandwiched between the first and second plastic layers and the first plastic layer is disposed directly against the at least one body part of the user,
wherein the propolis composition is configured to at least partially reduce at least one of perceived pain, swelling, inflammation and irritation in the at least one body part of the user when the first plastic layer is disposed directly against the at least one body part of the user.

17. The method of claim 16, wherein the one or more components of the propolis composition function as an adhesive for removably attaching the patch to the user in addition to at least partially reducing at least one of perceived pain, swelling, inflammation and irritation in the at least one body part of the user when the patch is applied to the at least one body part.

18. A method of manufacturing a patch having a propolis composition embedded therein for at least partially reducing at least one of perceived pain, swelling, inflammation and irritation in at least one body part of a user, the method comprising:
soaking at least one cloth layer in a propolis composition for a predetermined period of time to form at least one cloth layer having the propolis composition embedded therein;
disposing a first plastic layer immediately against a first side of the at least one cloth layer; and
disposing a second plastic layer immediately against a second side of the at least one cloth layer opposite the first side, such that the at least one cloth layer is sandwiched between the first and second plastic layers,
wherein the propolis composition is configured to at least partially reduce at least one of perceived pain, swelling, inflammation and irritation in the at least one body part of the user when the patch is disposed directly against the at least one body part of the user.

19. The method of claim 18, wherein one or more components of the propolis composition are configured to migrate through micropores in at least one of the at least one cloth layer, the first plastic layer and the second plastic layer.

20. The method of claim 19, wherein the one or more components of the propolis composition function as an adhesive for removably attaching the patch to the user in addition to at least partially reducing at least one of perceived pain, swelling, inflammation and irritation in the at least one body part of the user when the patch is applied to the at least one body part.
